(19) United States
(12) Patent Application Publication
(10) Pub. No.: US 2005/0069498 A1
(43) Pub. Date: Mar. 31, 2005

(54) Method for identifying metabotropic glutamate receptor agonists and potentiators

(76) Inventors: Christopher S. Baccei, San Marcos, CA (US); Daniel S. Lorrain, San Diego, CA (US)

Correspondence Address: MERCK AND CO., INC, P O BOX 2000, RAHWAY, NJ, 07065-0907, US

(21) Appl. No.: 10/955,982

(22) Filed: Sep. 30, 2004

Related U.S. Application Data
(60) Provisional application No. 60/507,212, filed on Sep. 30, 2003.

(51) Int. Cl. A61K 49/00

(52) U.S. Cl. 424/9.2

(57) ABSTRACT

The present invention is directed to a method of identifying compounds that activate an mGluR2 receptor and are useful in the treatment of neuopsychological disorders, such as schizophrenia, anxiety, panic disorder, and drug withdrawal.

### BACKGROUND OF THE INVENTION

This invention pertains to a method for identifying compounds which are mGluR2/3 agonists or mGluR2 potentiators. The compounds identified by this method are of value in the treatment of neuropsychological disorders, such as schizophrenia, anxiety, panic disorder, and drug withdrawal.

Ketamine and phencyclidine (PCP) administered to humans produce symptoms common to schizophrenia and have been used in rodents to model the behavioral and neurochemical abnormalities associated with schizophrenia. Upon acute challenge these drugs activate limbic forebrain release of dopamine (DA while decreasing the release of GABA. It is unclear which (if any) of these effects underlie the behavioral aberrations caused by these drugs, however, recent findings have focused on the importance of glutamate. Pretreating animals with a group II selective metabotropic glutamate receptor (mGluR2/3) agonist prevented PCP and ketamine-evoked glutamate release.

In addition to blocking forebrain glutamate release, mGluR2/3 agonists inhibit the firing of neurons within the locus coeruleus (LC) (Vandergriff and Rasmussen, Neuropharmacology, 1999, 38, 217-222) and thus may reduce norepinephrine (NE) release in terminal regions. Clinical evidence exists linking a hyperactive noradrenergic system to the pathophysiology of schizophrenia. First, atypical antipsychotics have a broad spectrum of activities including α-adrenergic antagonistic properties. Secondly, norepinephrine levels have been shown to be elevated in cerebral spinal fluid and plasma of drug-free patients, while post mortem evidence revealed an increase of norepinephrine in brain tissue taken from schizophrenic patients. Finally, blockade of α-adrenergic receptors in rodents has been shown to inhibit the motor stimulant effects of NMDA antagonists while also preventing the disruption of prepulse inhibition. Together these data suggest that decreasing noradrenergic neurotransmission may be beneficial for improving clinical outcome in schizophrenia.

In the present invention, microdialysis demonstrates that ketamine evoked-norepinephrine release and, alternatively, ketamine evoked-serotonin release, in the ventral hippocampus (VHipp) is reversed by systemic pretreatment with a known mGluR2/3 receptor agonist. More generally, this technique can be used to quantify the ability of test compounds to interact with mGluR2/3 receptors, and is therefore of value in identifying compounds which are mGluR2/3 agonists or mGluR2 potentiators. The compounds identified by this method are of value in the treatment of neuropsychological disorders, such as schizophrenia, anxiety, panic disorder, and drug withdrawal.

Previously, analysis of glutamate release was used to identify potential mGluR2/3 agonists or mGluR2 potentiators, but the variation from study to study was unacceptable (see e.g., Moghaddam, et al, Science, 281, 1349-1352 (1998)). The reproducibility of the method described herein is superior to the prior art methods for identifying mGluR2/3 agonists or mGluR2 potentiators.

### SUMMARY OF THE INVENTION

This invention relates to methods for identifying compounds useful in the treatment of neuopsychological disorders, such as schizophrenia, anxiety, panic disorder, and drug withdrawal. Embodiments of the instant invention are directed to methods for measuring the ability of a test compound to suppress ketamine-evoked norepinephrine release or ketamine evoked-serotonin release by activating mGluR2 receptors.

### BRIEF DESCRIPTION OF THE DRAWINGS

FIG. 1. Diagram of rat brain, showing general placement of the microdialysis probe into the ventral hippocampus (VHipp) part of the brain of a rat.

FIG. 2. Ketamine produces a dose dependent increase in VHipp norepinephrine (NE) release. This chart shows the time course of the norepinephrine elevation after ketamine challenge.

FIG. 3. A bar chart showing the area under each 120 min. curve of FIG. 2.

FIG. 4. An mGluR2/3 agonist (Compound A) dose dependently inhibits ketamine-evoked norepinephrine release and this effect is reversed by co-treatment with the group II mGlu receptor selective antagonist (Compound C). Data (mean±SEM) are expressed as the area under each respective 120 min time course. Numbers inside the bars represent the n numbers. Data (mean±SEM) are reported as percentage of baseline. P<0.05: Significantly greater or lower norepinephrine levels compared to vehicle controls as revealed by one-factor ANOVA followed by Dunnett's post hoc comparisons.

### DETAILED DESCRIPTION OF THE INVENTION

The present invention provides a method for measuring the ability of a compound to activate an mGluR2 receptor, comprising the steps of:
a) stereotaxically implanting a microdialysis intracranial probe into the ventral hippocampus part of the brain of a test animal;
b) administering the compound to the test animal;
c) collecting a fluid sample through the microdialysis intracranial test probe; and
d) measuring the presence of norepinephrine or serotonin in the sample, thereby measuring the ability of the compound to activate the mGluR2 receptor.

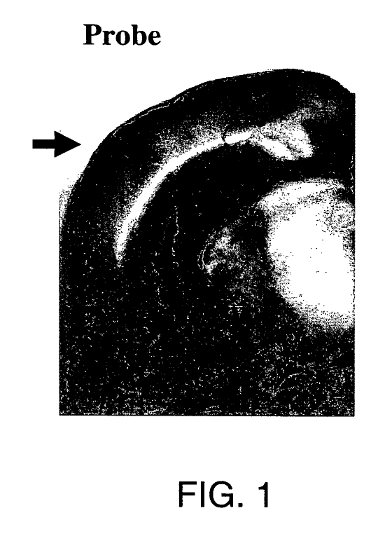

In this method, a microdialysis probe is stereotaxically implanted into the ventral hippocampus (VHipp) part of the brain of a test animal, such as a rat or other rodent. See FIG. 1 for an illustration of the general placement of the microdialysis probe in a rat brain. Fluid samples are withdrawn through the probe, and subjected to an analysis method to quantitate the amount of norepinephrine in the sample. Experiments can be performed using this general method to quantitate the effect of a test compound on the level of norepinephrine in the VHipp portion of the brain. Variations in norepinephrine levels in the VHipp are associated with neuropsychological disorders, so this method of general value in identifying compounds that can be used to treate neuropsychological disorders.

In one embodiment of the present invention, the presence of norepinephrine is quantified. In an alternate embodiment of the present invention, the presence of serotonin is quantified.

A further embodiment of this invention is a method for measuring the ability of a test compound to activate an mGluR2 receptor, comprising the steps of:
a. stereotaxically implanting a microdialysis intracranial probe into the ventral hippocampus part of the brain of:
   i. a set of test animals, and
   ii. a set of ketamine control animals;
b. administering the compound to the test animals;
c. administering ketamine to the test animals and to the ketamine control animals;
d. collecting fluid samples through the microdialysis intracranial probe of each set of animals;
e. quantifying the presence of norepinephrine or serotonin in the fluid samples from each set of animals; and
f. comparing the change in the level of norepinephrine release in the ventral hippocampus of the test animals to level of norepinephrine release in the ketamine control animals, or comparing the change in the level of serotonin release in the ventral hippocampus of the test animals to level of serotonin release in the ketamine control animals, respectively, thereby measuring the ability of the compound to activate the mGluR2 receptor.

Another embodiment of this invention comprises a method for measuring the ability of a test compound to activate an mGluR2 receptor, comprising the steps of:
a. stereotaxically implanting a microdialysis intracranial probe into the ventral hippocampus part of the brain of:
   i. a set of test animals,
   ii. a set of ketamine control animals, and
   iii. a set of vehicle control animals;
b. administering the compound to the test animals;
c. administering ketamine to the test animals and to the ketamine control animals;
d. collecting fluid samples through the microdialysis intracranial probe of each set of animals;
e. quantifying the presence of norepinephrine or serotonin in the fluid samples from each set of animals; and
f. comparing:
   i. the elevation of norepinephrine or serotonin release in the of ketamine control animals to the level of norepinephrine or serotonin, respectively, in the vehicle control animals, and
   ii. the suppression of norepinephrine or serotonin release, respectively, in the test animals compared to the ketamine control animals, thereby measuring the ability of the compound to activate the mGluR2 receptor.

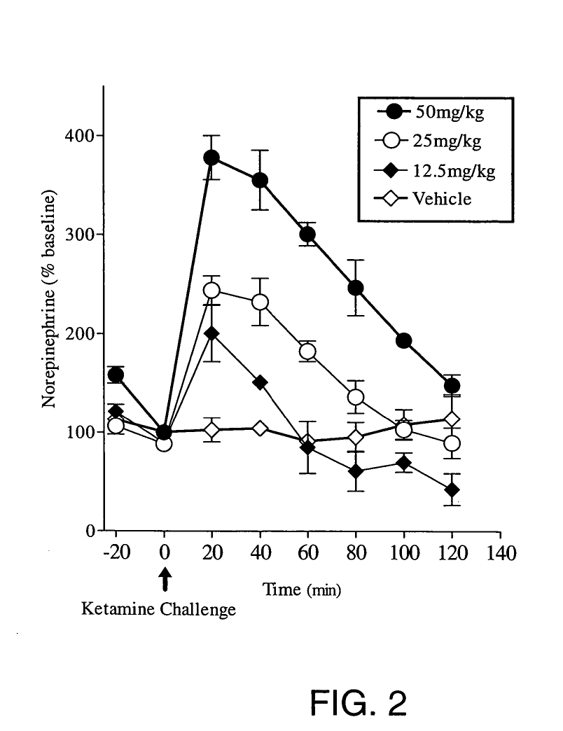

This embodiment of this invention employs three or more sets of animals, such as rats, in which an intracranial microdialysis probe is stereotaxically implanted into the VHipp portion of the brain. One set of animals is a vehicle control. A second set of animals is a ketamine control. A third set of animals is a test set. Optionally, there may be more than one set of test animals to measure dose response effects of the test compounds at different dosage levels, and more than one set of ketamine control animals to demonstrate that increased levels of ketamine increase the release of norepinephrine in the ventral hippocampus of the subject animals (see FIG. 2). Each set of animals in the test group, ketamine control group, and vehicle control groups comprises 1 to 20 animals, with 5 to 10 animals being a preferable group size that can demonstrate consistent results.

In an embodiment of the invention, the test set(s) of animals are pretreated with the test compound for a predetermined period of time. Then the test set(s) of animals and the ketamine control set of animals are challenged with ketamine, a compound known to elicit a strong norepinephrine and serotonin response in the VHipp. At predetermined intervals during the procedure, fluid samples from the VHipp are withdrawn throught the microdialysis probes. Norepinephrine levels are measured by a quantitative detection method, for example hplc with electrochemical detection. A chart is then constructed of the norepinephrine level in the vehicle controls, the ketamine controls, and the test set(s) of animals, with one axis being norepinephrine level, and the other axis being time. FIGS. 2-4 are examples of relevant charts.

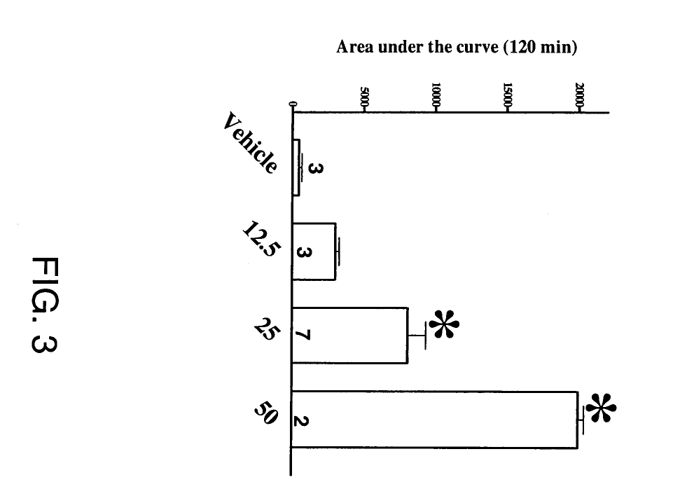

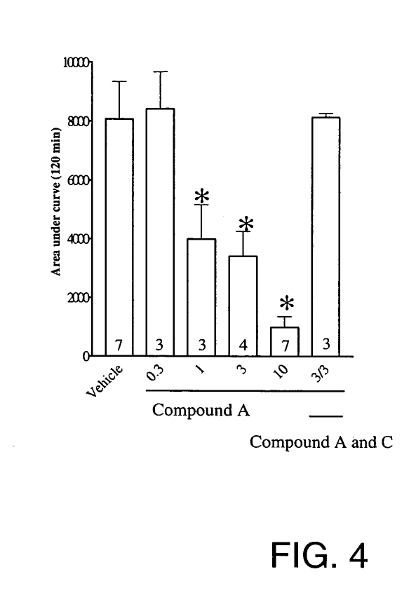

Elaborating on FIGS. 2-4, FIG. 2 shows results for three different dosage ranges of ketamine challenge and a vehicle control group, measured at 20 minute intervals over the course of 120 minutes. The Y axis shows norepinephrine levels. Over the course of the experiment, it is clearly seen that there is a dose-response effect of higher ketamine dosages showing a greater area under the curve (AUC) of norepinephrine release. The AUC levels are quantified in FIG. 3. For the test animals, administered compounds with potential mGluR2/3 agonist activity, a chart is constructed. Dose ranges of a known mGluR2 agonist (Compound A), plotted against norepinephrine levels indicate that escalating dosages of the mGluR2 agonist suppress and completely eliminate the elevated norepinephrine level seen in the control group following administration of ketamine. FIG. 4 summarizes this data. The vehicle animals AUC is also shown. This data shows that increased dosage levels of the mGluR2 agonist reduce the AUC of norepinephrine release. The last bar of FIG. 4 shows how this effect is reversed by co-treatment with a group II mGlu receptor selective antagonist (Compound C)—the AUC of norepinephrine is restored to the same level as the control and the lowest dose of the mGluR2 agonist.

In an embodiment of this invention, the quantitation of norepinephrine in the fluid withdrawn from the microdialysis probes is performed by HPLC coupled to electrochemical detection, but other techniques known to those skilled in the art may be used.

The value of this method, and the data derived therefrom, is in identifying compounds of value in the treatment of the neuropsychological disorders, wherein the compounds are an mGluR2/3 agonist or mGluR2 potentiators, and the disorder is, for example, schizophrenia, anxiety, panic disorder, and drug withdrawal. A secondary therapeutic indication is anesthetic induced sleep time. Each of the aforementioned disorders involves aberrant norepinephrine transmission. While the compounds used in the examples and illustrations are the known mGluR2 agonists, and the known mGluR2 antagonist, test compounds with suspected or unknown mGluR2/3 agonist or mGluR2 potentiator activity are used in the normal practice of this invention. By comparing the relative ability of test compounds to inhibit norepinephrine release in the rat VHipp, meaningful comparisons between compounds can be made, and the results from these studies will be an aide to selecting human therapeutics for the aforementioned neuropyschological disorders.

The following examples are provided so that the invention might be more fully understood. These examples are illustrative only and should not be construed as limiting the invention in any way.

### EXAMPLES

Subjects. Male Sprague-Dawley rats (250-300 g) were purchased from Harlan (San Diego, Calif.). All animals were maintained under a 12 h light/dark cycle with lights on at 0630 h and off at 1830 h. All microdialysis and behavioral experiments occurred during the light phase of the light/dark cycle. All procedures were performed in accordance with The Guide for the Care and Use of Laboratory Animals and were approved by the Institutional Animal Care and Use Committee. Before and after surgeries, rats were allowed free access to standard rat chow and water. Temperature and relative humidity were maintained at 22-24° C. and 50-55%, respectively.

Materials. Ketamine (in the form of Ketaset, 100 mg/ml) was obtained from Fort Dodge Animal Health (Fort Dodge, Iowa

Intracranial implants. Under 3% isoflurane (in 1% O₂) anesthesia, rats were implanted with a unilateral microdialysis probe CMA/11 with a 3 mm dialysis tip in the VHipp (anteroposterior, −5.3 mm; mediolateral, −4.6 mm; and dorsoventral, −7.5 mm) (Paxinos and Watson, 1998). The microdialysis probe was slowly lowered into position and was then fixed to the skull by means of 3 anchoring screws and application of dental acrylic. For microinjection experiments, animals additionally received a CMA/7 guide cannulae aimed at a specified region; LC (anteroposterior, −9.73 mm; mediolateral, −1.3 mm; and dorsoventral, −5.0 mm), mPFC (anteroposterior, +3.2 mm; mediolateral, −0.7 mm; and dorsoventral, −4.0 mm) or lateral ventricle (anteroposterior, −0.8 mm; mediolateral, +1.4 mm; and dorsoventral, −3.0 mm). Immediately following surgery rats were placed in their testing arena and allowed to recover for a minimum of 14 hours.

In vivo microdialysis and chromatography. For all microdialysis experiments, rats were tethered to a CMA awake animal system by means of a plastic collar (CMA Microdialysis, Acton, Mass.). All fluid connections were made using FEP tubing (internal volume of 1.2 μl/100 mm, CMA). Following the appropriate post-surgery recovery period (˜14 hours=˜0700 h) perfusion through the dialysis probe with artificial cerebral spinal fluid (aCSF; 145 mM NaCl; 2.7 mM KCl; 1.0 mM MgCl₂ and 1.2 mM CaCl₂; pH 7.4) was increased and maintained at 3.0 μl/min. A 2-hour stabilization period was then allowed prior to sample collection. Fractions were collected at 20-min intervals (60 μl) into 250 μl glass vials via a BAS honeycomb fraction collector maintained at 4° C. (BAS HoneyComb; BAS, West Lafayette, Iowa). Samples were subsequently analyzed for neurotransmitter content by HPLC (see details below) within a 24-hour period.

Norepinephrine (NE), serotonin (5-HT) and HVA were assayed by HPLC coupled to electrochemical detection. Samples were loaded into an ESA autosampler (model 465). The amines were separated on a C18 reverse phase column (Phenomenex Luna, 150×2.0 mm, 3 μm particles) and detected by an ESA Coulochem controller unit using a model 5041 microdialysis analytical cell fitted with a glassy carbon electrode set to oxidize at +250 mV. The mobile phase used was a phosphate buffer purchased from ESA (MD-TM). The rate of mobile phase flow through the system was 0.2 ml/min. NE, HVA and 5-HT eluted with a retention time of approximately 4.5, 10 and 16 min, respectively, under these isocratic conditions.

Data analysis. Dialysis data collected and analyzed were not corrected for in vitro probe recovery. Time course data represent the percent change from baseline (calculated by taking the average of two pretreatment samples). Summary data represent the raw area under each 120 min time course curve. Data were then subject to either a simple between subjects t-test or to a one-way ANOVA. Post hoc analyses were made using either Tukey's multiple comparisons test or Dunnett's method. Activity data (total distance traveled) represent the group mean (±SEM) recorded for the total duration of the 120 min test period. Data were analyzed by one-way ANOVA followed by Dunnett's or Student-Newman-Keuls tests. To examine the time course of drug effects, data are also expressed as the mean (±SEM) distance traveled recorded in 10 min intervals over the 120 min test period. These data were analyzed by two-way repeated measures ANOVA followed by Student-Newman-Keuls tests.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

1. A method for measuring the ability of a compound to activate an mGluR2 receptor, comprising the steps of:
a) stereotaxically implanting a microdialysis intracranial probe into the ventral hippocampus part of the brain of a test animal;
b) administering the compound to the test animal;
c) collecting a fluid sample through the microdialysis intracranial test probe; and
d) measuring the presence of norepinephrine or serotonin in the sample thereby measuring the ability of the compound to activate the mGluR2 receptor.

2. A method for measuring the ability of a compound to activate an mGluR2 receptor, comprising the steps of:
a) stereotaxically implanting a microdialysis intracranial probe into the ventral hippocampus part of the brain of:
i) a set of test animals, and
ii) a set of ketamine control animals;
b) administering the compound to the test animals;
c) administering ketamine to the test animals and to the ketamine control animals;
d) collecting fluid samples through the microdialysis intracranial probe of each set of animals;
e) quantifying the presence of norepinephrine or serotonin in the fluid samples from each set of animals; and
f) comparing the change in the level of norepinephrine release in the ventral hippocampus of the test animals to level of norepinephrine release in the ketamine control animals, or comparing the change in the level of serotonin release in the ventral hippocampus of the test animals to level of serotonin release in the ketamine control animals, respectively, thereby measuring the ability of the compound to activate the mGluR2 receptor.

3. The method of claim 2 wherein the compound has a modulatory effect on norepinephrine release.

4. The method of claim 2 wherein each set of test animals comprises a population of one or more rats.

5. The method of claim 4 further employing two or more sets of test animals, wherein each set of test animals is tested at a different dose of the test compound.

6. The method of claim 2 wherein the fluid samples collected through the microdialysis intracranial probe of each set of animals is collected at identical time intervals for each set of animals before, during, and after administration of ketamine and the test compound to each set of animals.

7. The method of claim 2 wherein the comparison of norepinephrine or serotonin release in the ventral hippocampus of each set of animals is made by means of a plot of data points of norepinephrine measurements from samples taken at identical time intervals for each set of animals, and the area under the curve of the curve of norepinephrine levels is compared.

8. The method of claim 2 wherein the presence of norepinephrine is quantified.

9. The method of claim 2 wherein the presence of serotonin is quantified.

10. A method for measuring the ability of a compound to activate an mGluR2 receptor, comprising the steps of:
a) stereotaxically implanting a microdialysis intracranial probe into the ventral hippocampus part of the brain of:
i) a set of test animals,
ii) a set of ketamine control animals, and
iii) a set of vehicle control animals;
b) administering the compound to the test animals;
c) administering ketamine to the test animals and to the ketamine control animals;
d) collecting fluid samples through the microdialysis intracranial probe of each set of animals;
e) quantifying the presence of norepinephrine or serotonin in the fluid samples from each set of animals; and
f) comparing:
i) the elevation of norepinephrine or serotonin release in the ketamine control animals to the level of norepinephrine or serotonin, respectively, in the vehicle control animals, and
ii) the suppression of norepinephrine or serotonin release, respectively, in the test animals compared to the ketamine control animals.
thereby measuring the ability of the compound to activate the mGluR2 receptor.

11. The method of claim 10 wherein each set of test animals comprises a population of one or more rats.

12. The method of claim 11 further comprising two or more sets of test animals, wherein each set of test animals is tested at a different dose of the test compound.

13. The method of claim 10 wherein the fluid samples collected through the microdialysis intracranial probe of each set of animals is collected at identical time intervals for each set of animals, and where time intervals are chosen before, during, and after administration of ketamine to the set of ketamine control animals and the test compound to the set of test animals.

14. The method of claim 10 wherein the comparison of norepinephrine or serotonin release in the ventral hippocampus of each set of animals is made by means of a plot of data points of norepinephrine measurements from samples taken at identical time intervals for each set of animals, and the area under the curve of the curve of norepinephrine levels is compared.

15. The method of claim 10 wherein the presence of norepinephrine is quantified.

16. The method of claim 10 wherein the presence of serotonin is quantified.

17. A method for identifying a compound useful for the treatment of neuropsychological disorders, comprising the method of claim 1.

18. The method of claim 17 wherein the compound useful for the treatment of neuropsychological disorders is an mGluR2/3 agonist or an mGluR2 potentiator.

19. The method of claim 18 wherein the neuropsychological disorder is selected from the group consisting of schizophrenia, anxiety, panic disorder, and drug withdrawal.
